(19) United States
(12) Patent Application Publication
(10) Pub. No.: US 2022/0135929 A1
(43) Pub. Date: May 5, 2022

(54) APPARATUS AND METHOD FOR ENHANCING ANAEROBIC DIGESTION BASED ON THE COUPLING OF ELECTRON TRANSFER WITH MICROBIAL ELECTROLYTIC CELL

(71) Applicant: Tongji University, Shanghai (CN)

(72) Inventors: Xiaohu DAI, Shanghai (CN); Lei LI, Shanghai (CN); Jun GAO, Shanghai (CN); Bin DONG, Shanghai (CN); Lingling DAI, Shanghai (CN)

(21) Appl. No.: 17/141,600

(22) Filed: Jan. 5, 2021

(30) Foreign Application Priority Data
Oct. 29, 2020 (CN) 202011184885.7

(51) Int. Cl. C12M 1/00; C12N 1/32; C12P 1/04; C12P 7/62

(52) U.S. Cl. CPC C12M 43/04; C12M 23/34; C12M 29/02; C12M 29/08; C12P 7/625; C12M 29/20; C12M 47/02; C12N 1/32; C12P 1/04; C12M 29/18

(57) ABSTRACT

Anaerobic digestion is enhanced based on the coupling of electron transfer with microbial electrolytic cell. A traditional anaerobic digestion reactor is used as the main body, a microbial electrolytic cell applied with a micro voltage is constructed, and the electron transfer in the system is optimized by an immobilized conductor material, to establish an efficient electron output-transfer-consumption anaerobic digestion pathway to produce methane.

### CROSS REFERENCE TO RELATED APPLICATION

The application claims priority to Chinese Application No. 202011184885.7, filed on Oct. 29, 2020, entitled “apparatus and method for enhancing anaerobic digestion based on the coupling of electron transfer with microbial electrolytic cell”, which is hereby incorporated by reference in its entirety.

### TECHNICAL FIELD

The present disclosure relates to a technical field of environmental protection and resource recovery, particularly relates to an apparatus and a method for enhancing anaerobic digestion based on the coupling of electron transfer with microbial electrolytic cell.

### BACKGROUND

With the development of economy and society and the rapid consumption of resources, environmental protection, green development, and resource recovery have become important issues around the world. A large amount of sewage and solid wastes are produced in human production and life. The recovery of resources and energy from these organic wastes has become a hot research topic. Anaerobic digestion, as a biological treatment technology widely used in the treatment of sewage, sludge and various organic wastes, makes it possible to realize the recovery of resources and energy while reducing, stabilizing these wastes, and making these wastes harmless, and becomes an important technical guarantee that supports the construction of ecological civilization and the sustainable development of society.

However, the anaerobic digestion requires a variety of microorganisms to complete a series of biochemical reactions, and the efficiency of anaerobic digestion is greatly limited by the unsmooth electron transfer and energy exchange between microorganisms. In terms of traditional anaerobic digestion, there are a series of shortcomings such as long reaction cycle, low organic matter degradation rate and low methane yield. Microbial electrolysis cell (MEC) enables the rate of biochemical reactions to be improved by forming biofilms on the anode and cathode to enhance the oxidation of organic matter at the anode and the reduction of carbon dioxide at the cathode, which is considered to be an effective method to improve the efficiency of anaerobic digestion. Both of patent applications entitled “method for enhancing methanogenesis in anaerobic digestion by coupling low-temperature thermal hydrolysis of surplus sludge with microbial electrolytic cell” (CN111574011A) and “anaerobic microbial electrochemical process with enhanced anaerobic digestion of sludge at the anode and enhanced reduction of carbon dioxide at the cathode” (CN109179938A) are based on this idea for technical research and development.

Nevertheless, there are still many shortcomings in the application of the above-mentioned microbial electrolytic cell in anaerobic digestion: biochemical reaction in the anode region and cathode region only accounts for a small portion of the entire anaerobic digestion system, and the electron transfer of the entire system cannot be improved by the applied micro-voltage alone; the problem that the electron transfer and energy exchange between microorganisms in the system are not smooth has not been fundamentally resolved, and the intermediate products such as volatile fatty acids sometimes are accumulated.

### SUMMARY

An objective of the present disclosure is to provide an apparatus and a method for enhancing anaerobic digestion based on the coupling of electron transfer with microbial electrolytic cell, so as to solve the problem of low efficiency of anaerobic digestion due to unsmooth electron transfer and energy exchange between microorganisms.

The objective of the present disclosure could be achieved with the following technical solutions:

An apparatus for enhancing anaerobic digestion based on the coupling of electron transfer with microbial electrolysis cell, comprising:

an anaerobic digestion reactor which is configured with an immobilized conductor material therein, and

a microbial electrolytic cell that is composed of a power source, a bioanode, and a biocathode,

wherein the immobilized conductor material is connected to the microbial electrolytic cell in such a way that the bioanode and the biocathode are respectively in full contact with the immobilized conductor material, to ensure an efficient interspecies electron transfer during the entire anaerobic digestion process.

In the apparatus according to the present disclosure, a traditional anaerobic digestion reactor is used as the main body, a microbial electrolytic cell applied with a micro voltage is constructed, and the electron transfer in the system is optimized by the immobilized conductor material, to establish an efficient electron output-transfer-consumption anaerobic digestion pathway to produce methane.

In some embodiments, the immobilized conductor material is formed by setting a conductor material with a good electrical conductivity and biocompatibility on a network structure.

In some embodiments, the conductor material includes one or more of graphene, carbon nanotube, graphite rod, graphite felt, carbon cloth, carbon brush, platinum carbon, and iron electrode. In some embodiments, the network structure includes titanium/titanium alloy mesh and iron/ferroalloy mesh, and the network structure has holes of 5 to 300 mesh. The conductor material can be immobilized, for example, in the form of a coating such as a graphene coating and a carbon nanotube coating, on the network structure.

In some embodiments, the power source is a direct-current power source, with a voltage of 0.1-1.2 V. While stimulating the electrical activity of microorganisms, the micro voltage optimizes the oxidation-reduction potential of the entire system, thereby enhancing the degradation of organic matter at the anode and the reduction of carbon dioxide at the cathode.

In some embodiments, the anaerobic digestion reactor is provided with a feed inlet in its middle, a non-gas phase outlet at its bottom, and a gas outlet at its top; the immobilized conductor material and the bioanode and biocathode of the microbial electrolytic cell are arranged in a reaction zone of the anaerobic digestion reactor, and the immobilized conductor material is arranged close to the feed inlet. The raw material fed for anaerobic digestion may fully contact with the immobilized conductor material near the feed inlet, improving the mass transfer of the system. The network structure further optimizes the adhesion effect of microorganisms, thereby improving the efficiency of electron transfer.

In some embodiments, the reaction zone of the anaerobic digestion reactor is provided with a stirring mechanism below the immobilized conductor material.

In some embodiments, paddle plates are staggered with each other on the stirring mechanism, and the middle surface of the paddle plates are roughened and made porous, and are covered with a conductive coating, to further enhance the adhesion of microorganisms and electron transfer in the reaction zone.

In some embodiments, the stirring mechanism has a stirring rate of 60-150 rpm, and the stirring mechanism is paused for 0.5-10 minutes after every stirring for 0.5-2 minutes. A stirring with a suitable stirring rate may be to improve the mixing of materials and mass transfer effects of the system, without destroying the aggregation of microorganisms and the binding of microorganisms with the substrate.

In some embodiments, a physicochemical index sensor is arranged inside the reaction zone of the anaerobic digestion reactor, and the physicochemical index sensor is to monitor the change of the physicochemical index during the anaerobic digestion, including pH, oxidation-reduction potential (ORP), and electrical conductivity (EC). By monitoring these physicochemical indexs, it is possible to have a real-time understanding of the operation of the system, biochemical reactions, and electron transfer situation. The operating parameters may be adjusted and optimized in real time by combining the monitoring of physicochemical indexs and the monitoring of gas components.

In some embodiments, a gas sensor is arranged in a headspace zone above the reaction zone of the anaerobic digestion reactor. The gas sensor is to monitor the gas components in the biogas, including methane, carbon dioxide, and hydrogen sulfide. By monitoring the gas components, the gas production situation of the system and the change of methane proportion may be judged to determine the operating effect of the system.

A method for enhancing anaerobic digestion based on the coupling of electron transfer with microbial electrolytic cell, comprising,

arranging an immobilized conductor material in a reaction zone of an anaerobic digestion reactor, wherein the immobilized conductor material is formed by setting a conductor material with a good electrical conductivity and biocompatibility on a network structure;

connecting the immobilized conductor material to a microbial electrolytic cell that is composed of a direct-current power source, a bioanode, and a biocathode in such a way that the bioanode and biocathode are respectively in full contact with the immobilized conductor material; and

forming a closed-loop electron pathway during the reaction, by performing an oxidative decomposition reaction of organic matter at the bioanode, and a reduction reaction of carbon dioxide at the biocathode, making the material fed into the anaerobic digestion reactor fully contact with the immobilized conductor material, to ensure an efficient interspecies electron transfer during the entire anaerobic digestion process.

According to the biogas production for the whole system and the proportion of methane in the biogas, it is possible to adjust the voltage provided by the direct-current power source and the immobilized position of the conductor material to optimize the electron transfer and biochemical reaction effects, and to improve the efficiency of the anaerobic digestion system.

The apparatus and the process using the same are suitable for anaerobic biological treatment of sewage, sludge and other organic wastes (food waste, kitchen waste, livestock manure, straw). The apparatus is heated by using a jacketed water bath, and is operated at a temperature of 25-65° C.

A principle of the present disclosure is, considering that the process of anaerobic digestion to produce methane involves the mass transfer of acid-producing bacteria and methanogenic archaea, and interspecies electron transfer, to construct a direct interspecies electron transfer (DIET) during the anaerobic digestion process by microbial culture or the addition of the exogenous conductor material, to form a high-efficiency electron transfer pathway, thereby improving the efficiency of anaerobic digestion. The anode and cathode of the microbial electrolytic cell, which also may be made of the conductor material, are coupled with the immobilized conductive material in the system, thereby forming an efficient output-transfer-consumption electron pathway in the entire anaerobic system, enhancing the degradation of organic matter and improving the efficiency of methane production.

Compared with the prior art, the present disclosure has the following advantages:

(1) the present disclosure proposes for the first time an apparatus and a method for enhancing anaerobic digestion based on the coupling of electron transfer with microbial electrolytic cell, and makes it possible to construct a high-efficiency electron output-transfer-consumption anaerobic digestion pathway to produce methane;

(2) the present disclosure makes it possible to overcome bottleneck problem of the traditional anaerobic digestion, for example, long cycle period and low methane yield, improve the efficiency of anaerobic digestion, promote to smooth the progress of anaerobic digestion to produce methane, and enhance the degradation of organic matter while increasing the methane production rate and the proportion of methane in the biogas;

(3) the electrical energy consumption incurred by micro voltage is far less than the electrical energy brought by the increased methane production. The immobilized conductor material used is to achieve a stable effect during the anaerobic digestion process and is not easy to be lost; it can be reused to improve the efficiency of anaerobic digestion while reducing cost, with good economic benefits;

(4) the method and apparatus according to the present disclosure are suitable for a low-temperature, medium-temperature, and high-temperature anaerobic digestion system, with a broad application range, clear technical effects, and good application prospects.

### BRIEF DESCRIPTION OF THE DRAWINGS

FIG. 1 shows a schematic diagram of a structure of the anaerobic digestion apparatus according to an embodiment of the present disclosure.

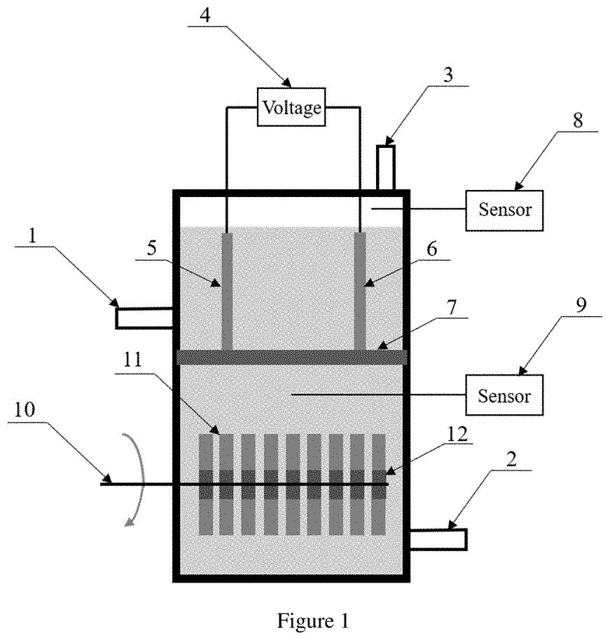

### DETAILED DESCRIPTION OF THE EMBODIMENTS

The present disclosure is illustrated in detail below with reference to the accompanying FIGURE and examples.

### Example 1

This example aims to illustrate an apparatus for enhancing anaerobic digestion based on the coupling of electron transfer with microbial electrolytic cell and its specific operation steps.

The FIGURE shows an apparatus for enhancing anaerobic digestion based on the coupling of electron transfer with microbial electrolytic cell, in which a feed inlet 1 is at the middle of the apparatus and a non-gas phase outlet 2 is at a lower thereof, and a gas outlet 3 is at the top of the apparatus which may be in communication with a subsequent biogas purification device or collection device; a power supply device 4 is connected with an anode region 5 and a cathode region 6 via wires, and the anode region is close to the feed inlet and the cathode region is close to the gas outlet, thereby enhancing the oxidation and decomposition of the fed organic matter at the anode and the reduction of carbon dioxide at the cathode; both the anode and cathode conductor materials are in full contact with an immobilized conductor material 7 to form a closed-loop electron pathway; a gas sensor 8 is arranged in the headspace zone of the apparatus and a physiochemical index sensor 9 is arranged inside the reaction zone, to realize online real-time monitoring of both the gas phase and the liquid phase; a stirring mechanism 10 is arranged in the middle and lower of the apparatus, to improve the mixing of materials and mass transfer effects of the system; paddle plates 11 are staggered with each other on the stirring mechanism; the middle surfaces of the paddle plates are roughened and made porous, and are covered with a conductive coating 12, to further enhance the adhesion of microorganisms and electron transfer in the reaction zone.

An apparatus for enhancing anaerobic digestion based on the coupling of electron transfer with microbial electrolytic cell was used, and its specific operation steps were as follows:

S1. The pH, total solid (TS) content and volatile solid (VS) content of the anaerobic digestion inoculum were tested, and an appropriate amount of the inoculum was inoculated into the reactor according to the operating conditions.

S2. The pH, total solid content and volatile solid content of the materials to be digested by anaerobic digestion were tested, and the materials were fed into the reactor through the feed pipe in the feed inlet, a temperature control device was started to heat and the stirring mechanism was started, the gas sensor and the physiochemical index sensor were turn on, the pH of the feed material was adjusted according to the feedback results of the sensor, and the power supply device was turn on when suitable.

S3. The voltage was adjusted in real time according to the system conductivity and oxidation-reduction potential. After the gas production of the system was stable, the stirring rate was adjusted, the biogas production and the methane proportion were recorded, a system model of input voltage and methane production was established, the organic load of the system was adjusted, to optimize the efficiency of methane production in the system.

S4. According to the conditions of system batch operation, semi-continuous operation or continuous operation, the non-gas phase outlet was adjusted to be open or closed, and the pH, total solid (TS) content and volatile solid (VS) content of the discharged materials were tested, and a portion of the discharged materials could be recycled as the inoculum.

In an actual application of this apparatus, the unpretreated materials, or the pretreated or pre-fermented materials from the preceding reactor may be directly fed through the feed inlet, both of which enable the efficient electron transfer and anaerobic methane production to be realized.

During the operation of the apparatus, electroactive biofilm was gradually formed on the surfaces of the anode, cathode and immobilized conductive material, which is to improve the stability and the efficiency of the system, thus gradually increasing the organic load of the system.

In order to meet the actual production needs, the apparatus can be set in series or parallel. When set in series, the discharged materials from a preceding reactor can be used as the feed materials for a subsequent reactor, and the organic load is gradually reduced, which can further increase the degradation rate of the organic matter and methane production in the anaerobic digestion. When set in parallel, simultaneous anaerobic digestion in multiple reactors can be realized by arranging one set of power supply device alone.

### Example 2

This example aims to implement a method for enhancing anaerobic digestion based on the coupling of electron transfer with microbial electrolytic cells in a semi-continuous anaerobic digestion experiment with food waste as the substrate.

Food waste (VS/TS=61.7-93.9%, TS=9.6-25.3%) after removing bones and uniformly crushing was used as the substrate, the digested sludge (VS/TS=33.1-47.6%, TS=1.9-6.7%) from the sludge anaerobic digestion reactor that operated stably was used as the inoculum, and an anaerobic digestion experiment was carried out in the apparatus according to the present disclosure with a working volume of 4 L.

The apparatus was operated semi-continuously, with a daily feed and discharge of 200 mL, and sludge retention time (SRT) of 20 days. The anaerobic digestion was carried out at 37° C. while stirring, and the stirring was paused for 3 minutes after every stirring for 1 minute, with a stirring rate of 80 r/min. During the experiment, the pH, ORP, and EC of the system were monitored, the TS and VS contents of the fed and discharged materials were measured, and the biogas production and the proportion of methane in the biogas were recorded.

### Comparative Example 1

This example was performed as described in Example 2, except that an ordinary anaerobic digestion reactor was used instead of the apparatus according to the present disclosure.

### Comparative Example 2

This example was performed as described in Example 2, except that an ordinary microbial electrolytic cell was used instead of the apparatus according to the present disclosure.

Compared with Comparative Example 1, Comparative Example 2 exhibited that the methane production rate was increased, but the system was unstable and the volatile fatty acids were accumulated. Compared with Comparative Example 1 or Comparative Example 2, Example 2 exhibited that both the methane production rate and the degradation rate of the organic matter were further increased, wherein the maximum methane production rate in the system of Example 2 was increased to 130.58 mL/(g VSadd·d), respectively from 88.89 mL/(g VSadd·d) of Comparative Example 1, and 109.91 mL/(g VSadd·d) of Comparative Example 2, which was increased by 47% and 19% respectively in relative to that of Comparative Example 1 and Comparative Example 2, and the degradation rate of organic matter in Example 2 was increased to 70.1% respectively from 48.9% of Comparative Example 1, and 56.2% of Comparative Example 2, which was increased by 30% and 25% respectively in relative to that of Comparative Example 1 and Comparative Example 2.

### Example 3

This example aims to implement a method for enhancing anaerobic digestion based on the coupling of electron transfer with microbial electrolytic cell in a continuous-flow anaerobic digestion experiment with sludge as the substrate.

Surplus sludge (VS/TS=47.9-69.1%, TS=1.8-6.1%) from the secondary sedimentation tank was used as the substrate, the digested sludge (VS/TS=35.1-47.6%, TS=1.9-6.5%) from the sludge anaerobic digestion reactor that operated stably was used as the inoculum, and a continuous-flow methane production experiment was conducted in a reactor with a working volume of 8 L.

The apparatus was continuously operated at 37° C. while stirring, and the stirring was paused for 1 minute after every stirring for 1 minute, with a stirring rate of 100 r/min. During the experiment, the pH, ORP, and EC of the system were monitored, the TS and VS contents of the fed and discharged materials were measured, and the biogas production and methane proportion in the biogas were recorded.

### Comparative Example 3

The example was performed as described in Example 3, except that an ordinary anaerobic digestion reactor was used instead of the apparatus according to the present disclosure.

### Comparative Example 4

The example was performed as described in Example 3, except that an ordinary microbial electrolytic cell was used instead of the apparatus according to the present disclosure.

Compared with Comparative Example 3, Comparative Example 4 exhibited that the methane production was slightly increased, but the proportion of methane in biogas did not change significantly. Compared with Comparative Example 3 or Comparative Example 4, Example 3 exhibited that both the methane production and the proportion of methane in biogas were further increased, wherein the daily methane production of the system in Example 3 was increased to 121.03 mL/g VS_{add} respectively from 83.91 mL/g VS_{add} of Comparative Example 3 and 97.79 mL/g VS_{add} of Comparative Example 4, which increased by 44% and 24% respectively in relative to that of Comparative Example 3 and Comparative Example 4, and the proportion of methane in biogas in Example 3 was increased to 82.1% respectively from 68.9% of Comparative Example 3 and 69.1% of Comparative Example 4, and was increased by 19% in relative to that of Comparative Example 3 and Comparative Example 4.

The above description of the embodiments is to help those skilled in the art understand and use the disclosure. Those skilled in the art can obviously easily make various modifications to these embodiments and apply the general principles as described here to other embodiments without creative labour. Therefore, the present disclosure is not limited to the above-mentioned embodiments. The improvements and modifications made by those skilled in the art based on the present disclosure without departing from the scope of the present disclosure should fall within the protection scope of the present disclosure.

1. An apparatus for enhancing anaerobic digestion based on the coupling of electron transfer with microbial electrolysis cell, comprising:
an anaerobic digestion reactor which is configured with an immobilized conductor material therein, and
a microbial electrolytic cell that is composed of a power source, a bioanode and a biocathode,
wherein the immobilized conductor material is connected to the microbial electrolytic cell in such a way that the bioanode and the biocathode are respectively in full contact with the immobilized conductor material.

2. The apparatus for enhancing anaerobic digestion based on the coupling of electron transfer with microbial electrolysis cell as claimed in claim 1, wherein the immobilized conductor material is formed by setting a conductor material with a good electrical conductivity and biocompatibility on a network structure.

3. The apparatus for enhancing anaerobic digestion based on the coupling of electron transfer with microbial electrolysis cell as claimed in claim 2, wherein the conductor material includes one or more of graphene, carbon nanotube, graphite rod, graphite felt, carbon cloth, carbon brush, platinum carbon, and iron electrode;
the network structure includes titanium/titanium alloy mesh and iron/ferroalloy mesh, and the network structure has holes of 5 to 300 mesh.

4. The apparatus for enhancing anaerobic digestion based on the coupling of electron transfer with microbial electrolysis cell as claimed in claim 1, wherein the power source is a direct-current power source, with a voltage of 0.1-1.2 V.

5. The apparatus for enhancing anaerobic digestion based on the coupling of electron transfer with microbial electrolysis cell as claimed in claim 1, wherein the anaerobic digestion reactor is provided with a feed inlet in its middle, a non-gas phase outlet at its bottom, and an gas outlet at its top, the immobilized conductor material and the bioanode and biocathode of the microbial electrolytic cell are arranged in a reaction zone of the anaerobic digestion reactor, and the immobilized conductor material is arranged close to the feed inlet.

6. The apparatus for enhancing anaerobic digestion based on the coupling of electron transfer with microbial electrolysis cell as claimed in claim 5, wherein the reaction zone of the anaerobic digestion reactor is provided with a stirring mechanism below the immobilized conductor material.

7. The apparatus for enhancing anaerobic digestion based on the coupling of electron transfer with microbial electrolysis cell as claimed in claim 6, wherein paddle plates are staggered with each other on the stirring mechanism, and the middle surface of the paddle plates are roughened and made porous, and are covered with a conductive coating.

8. The apparatus for enhancing anaerobic digestion based on the coupling of electron transfer with microbial electrolysis cell as claimed in claim 6, wherein the stirring mechanism has a stirring rate of 60-150 rpm, and the stirring mechanism is paused for 0.5-10 minutes after every stirring for 0.5-2 minutes.

9. The apparatus for enhancing anaerobic digestion based on the coupling of electron transfer with microbial electrolysis cell as claimed in claim 5, wherein a physicochemical index sensor is arranged inside the reaction zone of the anaerobic digestion reactor, and a gas sensor is arranged in a headspace zone above the reaction zone.

10. A method for enhancing anaerobic digestion based on the coupling of electron transfer with microbial electrolytic cell, comprising:
arranging an immobilized conductor material in a reaction zone of an anaerobic digestion reactor, wherein the immobilized conductor material is formed by setting a conductor material with a good electrical conductivity and biocompatibility on a network structure;
connecting the immobilized conductor material to a microbial electrolytic cell that is composed of a direct-current power source, a bioanode, and a biocathode in such a way that the bioanode and biocathode are respectively in full contact with the immobilized conductor material; and
forming a closed-loop electronic pathway during the reaction by performing an oxidative decomposition reaction of organic matter at the bioanode, and a reduction reaction of carbon dioxide at the biocathode, making the material fed into the anaerobic digestion reactor fully contact with the immobilized conductor material, to ensure an efficient interspecies electron transfer during the entire anaerobic digestion process.
